(19) United States
(12) Patent Application Publication
(10) Pub. No.: US 2020/0368262 A1
(43) Pub. Date: Nov. 26, 2020

(54) Use of Timosaponin Enzymatically Transformed Product in Preparation of Drugs for Inhibiting Skin Superficial Fungi

(71) Applicant: Shanghai University of Traditional Chinese Medicine, Shanghai (CN)

(72) Inventors: Tong ZHANG, Shanghai (CN); Yue DING, Shanghai (CN); Lu LU, Shanghai (CN); Sheng'an ZHANG, Shanghai (CN); Min JIANG, Shanghai (CN); Fulun LI, Shanghai (CN)

(21) Appl. No.: 16/848,708

(22) Filed: Apr. 14, 2020

(30) Foreign Application Priority Data
May 20, 2019 (CN) 201910419861.6

(51) Int. Cl. A61K 31/7048; A61K 36/8964; A61P 31/10; A61P 17/00; C12P 33/20

(52) U.S. Cl. CPC A61K 31/7048; A61K 36/8964; A61P 31/10; A61K 2236/333; C12P 33/20; A61K 2236/19; A61P 17/00

(57) ABSTRACT

The present disclosure provides use of a timosaponin enzymatically transformed product in preparation of drugs for inhibiting skin superficial fungi. In the use provided in the present disclosure, the timosaponin enzymatically transformed product is prepared by enzymatically transforming rhizoma anemarrhenae extract with β-glucosidase or a compound enzyme of xylanase and cellulose after purification. In the present disclosure, as an active pharmaceutical ingredient for inhibiting skin superficial fungi, the timosaponin enzymatically transformed product has the minimum inhibitory concentration of 4 mg/L against fungi, which is obviously superior to total timosaponin in the bacteriostatic capability, and has a certain application prospect in the aspect of treating skin superficial mycoses.

### CROSS-REFERENCE TO RELATED APPLICATION

The present disclosure claims the priority to the Chinese patent application with the filing number 201910419861.6 filed on May 20, 2019 with the Chinese Patent Office, and entitled “Use of Timosaponin Enzymatically Transformed Product in Preparation of Drugs for Inhibiting Skin Superficial Fungi”, which is incorporated herein by reference in entirety.

### TECHNICAL FIELD

The present disclosure relates to the field of drug, in particular to use of a timosaponin enzymatically transformed product and a main component timosaponin AIII thereof in preparation of drugs for inhibiting skin superficial fungi.

### BACKGROUND ART

Superficial mycoses are a class of diseases caused by infection of superficial tissues such as hair, skin, nails and toenails of human body by pathogenic fungi or conditionally pathogenic fungi. The prevalence of superficial mycoses is reported to be 20%˜25% in domestic populations, with an increasing trend of incidence year by year. The clinical common pathogenic or conditional fungi mainly include trichophyton rubrum, trichophyton mentagrophytes, trichophyton purpurea, candida albicans, cryptococcus neoformans, microsporum gypseum, chromoblastomyces and so on. These pathogenic or conditional fungi can cause diseases such as onychomycosis, tinea manuum, tinea pedis, tinea capitis, tinea corporis, tinea cruris, and onychomycosis, and these diseases have a long disease course, recur easily and can be developed into secondary infections, thus severely affecting the health, work, social contact and daily life of patients. The sleep, work or life of most patients is seriously influenced by symptoms such as pruritus.

At present, the prevention and treatment of skin superficial fungal infection diseases in clinic mainly adopts chemical drug preparations, including imidazole drugs represented by imazalil, econazole, miconazole, and ketoconazole, and allylamine antifungal drugs represented by naftifine, terbinafine and butenafine. However, these chemical drug preparations easily cause problems such as poor patient compliance because of long treatment course or local discomfort caused by coated drug, and also may cause problems such as poor treatment effect and high recurrence rate due to factors such as poor local drug permeability. With the wide application of antifungal drugs, the drug resistance against fungi is increasing. The demand for antifungal drugs is rapidly increased, and to find a novel and efficient drug, with low toxicity and good selectivity, for resisting superficial fungal infection becomes a quite urgent subject in the medical field.

China has various kinds of Chinese herbal drugs with rich resources, and the traditional Chinese herbs have particular advantages and plays an important role in the aspect of preventing skin diseases. Rhizoma anemarrhenae, dried rhizome of Liliaceae plant Anemarrhena asphodeloides Bge., has bitter taste and cold nature, enters lung, stomach and kidney channels, and has effects of clearing heat-fire, nourishing yin and moistening dryness. Rhizoma anemarrhenae is rich in steroid saponins, xanthones, flavones, lignanoids, polysaccharides, organic acids, trace elements and other ingredients, wherein the steroid saponins are main ingredients, with a content of about 6% in the rhizome, and various varieties.

A lot of modern pharmacological research is carried out on timosaponin by modern pharmacology, and the rhizoma anemarrhenae is found to have pharmacological effects such as improving memory, inhibiting platelet aggregation, resisting oxidation and reducing blood sugar, etc. In addition, it has been reported that the total timosaponin (total saponin of rhizoma anemarrhenae) has potential effect of resisting skin fungi. However, the minimum inhibitory concentrations (MIC) of the total timosaponin against three kinds of dermatophytes (trichophyton gypseum, microsporum gypseum and epidermophyton floccosum) are 97.7, 781.3 and 781.3 mg/L, respectively, and the bacteriostatic effect is still unsatisfactory. However, in our laboratory, it has been found that the bacteriostatic capability of the total timosaponin is enhanced after having undergone enzymatic transformation with β-glucosidase or a compound enzyme of xylanase and cellulase, and the effect is comparable to that of clinical drugs posaconazole and fluconazole. In addition, the enzymatically transformed product of timosaponin has no irritation on skin.

Meanwhile, on the basis of the previously established enzymolysis process for hydrolyzing the total timosaponin with β-glucosidase, it is further optimized and studied, that is, the enzymolysis process of the total timosaponin is optimized using a compound enzyme of xylanase and cellulase instead of β-glucosidase because the former are cheaper and more available in raw materials. Furthermore, the enzymatic transformation rate of the timosaponin BII into AIII is improved. Meanwhile, the purification process of AIII from enzymatically transformed product is simplified, and has been verified through a pilot-scale production. The requirement of raw materials of the timosaponin enzymatically transformed product in preparation of can be met.

In view of this, the present disclosure is specifically proposed.

### SUMMARY

Embodiments of the present disclosure provide use of a timosaponin enzymatically transformed product in preparation of drugs for inhibiting skin superficial fungi, wherein the timosaponin enzymatically transformed product is prepared by enzymatically transforming rhizoma anemarrhenae extract with β-glucosidase to obtain a timosaponin enzymatically transformed product.

Embodiments of the present disclosure further provide use of timosaponin enzymatically transformed product containing high-purity timosaponin AIII in preparation of drugs for inhibiting skin superficial fungi, wherein the timosaponin enzymatically transformed product is prepared byenzymatically transforming rhizoma anemarrhenae extract with a compound enzyme of xylanase and cellulose, to obtain the timosaponin enzymatically transformed product after stirring to successfully with an alkaline solution and high concentration ethanol to remove impurities.

Embodiments of the present disclosure further provide a drug for inhibiting skin superficial fungi, wherein raw materials of the drug for inhibiting skin superficial fungi include a timosaponin enzymatically transformed product or timosaponin AIII.

### DETAILED DESCRIPTION OF EMBODIMENTS

Embodiments of the present disclosure will be described in detail below in combination with examples, while technicians in the field will understand that the following embodiments are merely for illustrating the present disclosure, but should not be considered as limitation to the scope of the present disclosure. If no specific conditions are specified in the examples, they are carried out under normal conditions or conditions recommended by manufacturers. If manufacturers of reagents or apparatuses used are not specified, they are conventional products commercially available.

In view of the potential application prospect of timosaponin in the aspect of bacteriostasis, and shortcomings of total timosaponin in the bacteriostatic performance, the present disclosure specifically provides use of inhibiting drugs with a timosaponin enzymatically transformed product as effective substance, and a method for preparing the effective substance, which is promising to realize low-dose treatment of superficial mycoses.

In one aspect, the present disclosure provides use of timosaponin enzymatically transformed product in preparation of drugs for inhibiting skin superficial fungi, wherein the timosaponin enzymatically transformed product is prepared by enzymatically transforming rhizoma anemarrhenae extract with 3-glucosidase to obtain the timosaponin enzymatically transformed product.

Timosaponin BII and timosaponin AIII are two important steroidal saponins in rhizoma anemarrhenae, wherein timosaponin BII is a main effective ingredient in rhizoma anemarrhenae with a content of up to 10%, while timosaponin AIII is at very low level in rhizoma anemarrhenae. The content of timosaponin AIII is only about 1/20 that of timosaponin BII. Research on the in vivo metabolic process of timosaponin BII shows that timosaponin BII transformed timosaponin AIII in vivo after loss of one molecule of sugar. On this basis, the present disclosure adopts an in vitro biological enzymolysis technology to enzymatically transform the rhizoma anemarrhenae extract to obtain an enzymatically transformed product with timosaponin AIII as the main ingredient. The bacteriostatic effect of the enzymatically transformed product is obviously superior to that of the rhizoma anemarrhenae extract and total timosaponin used in current researches, which shows a promissing application and provides alternative ways for treatment of skin superficial mycoses.

In some embodiments of the present disclosure, the rhizoma anemarrhenae extract is mainly prepared by: extracting rhizoma anemarrhenae with a solvent to obtain the rhizoma anemarrhenae extract;

Preferably, the rhizoma anemarrhenae includes one or two of medicinal material rhizoma anemarrhenae and rhizoma anemarrhenae decoction pieces;

Preferably, the solvent used for extracting rhizoma anemarrhenae includes: ethanol, aqueous ethanol, water and other conventional extraction solvents.

In some more preferred embodiments of the present disclosure, it further includes: after extracting rhizoma anemarrhenae with a solvent, concentrating and drying the resultant extracting solution, to obtain rhizoma anemarrhenae extract existing in a form of extractum or dry powder.

In some embodiments of the present disclosure, the step of enzymatically transforming rhizoma anemarrhenae extract with β-glucosidase includes:

incubating the rhizoma anemarrhenae extract and β-glucosidase in a solution to obtain the timosaponin enzymatically transformed product;

Preferably, the enzyme activity of the β-glucosidase is: 10-100 U/mg.

In some preferred embodiments of the present disclosure, the solution used for incubation is a buffer solution;

Preferably, the solution used for incubation includes: an acetic acid-sodium acetate buffer solution;

More preferably, pH of the buffer solution ranges from 3.79 to 4.95 (for example, but not limited to, 4.0, 4.2, 4.5, 4.7, etc.).

In some preferred embodiments of the present disclosure, the incubation is carried out at a temperature of 35-55° C. (for example, it may be, but not limited to, 40, 45, 50° C., etc.) for 1-3 h (for example, it may be, but not limited to, 2 h, etc.).

In some preferred embodiments of the present disclosure, the content of timosaponin AIII is 40% or more in the timosaponin enzymatically transformed product.

In some more preferred embodiments of the present disclosure, the step of enzymatically transforming rhizoma anemarrhenae extract with β-glucosidase includes:

incubating the rhizoma anemarrhenae extract and β-glucosidase in an acetic acid-sodium acetate buffer solution and drying the resultant enzymolysis product to obtain a timosaponin enzymatically transformed product;

In this embodiment, what is obtained is the timosaponin enzymatically transformed product wherein timosaponin AIII is the effective ingredient, with a content about 40%, and the content of timosaponin BII is less than 1%.

In some preferred embodiments of the present disclosure, the timosaponin enzymatically transformed product can be used for inhibiting one or more skin superficial fungi of candida albicans, cryptococcus neoformans, trichophyton rubrum, trichophyton mentagrophytes, and microsporum gypseum. Thus,the timosaponin enzymatically transformed product can be further used as an active ingredient for preparation of drugs for skin superficial fungi, and for treatment of superficial mycoses such as onychomycosis, tinea manuum, tinea pedis, tinea capitis, tinea corporis, tinea cruris, and onychomycosis.

In some preferred embodiments of the present disclosure, the timosaponin enzymatically transformed product can be used as an active ingredient, and mixed with a cosolvent to obtain corresponding pharmaceutical dosage forms, such as lotion, liniment, ointment and film coating agent.

In another aspect, the present disclosure provides use of a timosaponin enzymatically transformed product containing high-purity timosaponin AIII in preparation of drugs for inhibiting skin superficial fungi. Timosaponin BII and timosaponin AIII are two important steroidal saponins in rhizoma anemarrhenae. Timosaponin BII is the main effective ingredient in rhizoma anemarrhenae with a content of up to 10%, while timosaponin AIII is at a very low level in rhizoma anemarrhenae, which is only about 1/20 of timosaponin BII. Research on the in vivo metabolic process shows that timosaponin BII one molecule of sugar can be removed from timosaponin BII in vivo to transform into timosaponin AIII by loss of one molecule of sugar. On this basis, the present disclosure adopts an in vitro biological enzymolysis technology (a compound enzyme of xylanase and cellulase) to enzymatically transform the timosaponin extract to obtain an enzymatically transformed product with timosaponin AIII as a main ingredient. The bacteriostatic effect of the enzymatically transformed product is obviously superior to that of the timosaponin extract and total timosaponin used in current research, which shows a promissing application prospect, and provides alternative ways for treatment of skin superficial mycoses.

In the above, the method for preparation of the timosaponin enzymatically transformed product is: enzymatically transforming rhizoma anemarrhenae extract with a compound enzyme of xylanase and cellulose, to obtain the timosaponin enzymatically transformed product after stirring successfully with an alkaline solution and high concentration ethanol to remove impurities. The content of timosaponin AIII in enzymatically transformed product to 65%. In the process, the extraction rate of the timosaponin BII is 80%-85%, the enzymolysis transformation rate of AIII is 75.0%-80.0%, and the yield is 4550%.

In some embodiments of the present disclosure, the rhizoma anemarrhenae extract is mainly prepared by: extracting rhizoma anemarrhenae with a solvent to obtain the rhizoma anemarrhenae extract;

Preferably, the rhizoma anemarrhenae includes one or two of medicinal material rhizoma anemarrhenae and rhizoma anemarrhenae decoction pieces;

Preferably, the solvent used for extracting rhizoma anemarrhenae includes ethanol, aqueous ethanol, water and other conventional extraction solvents, preferably 50% ethanol.

In some more preferred embodiments of the present disclosure, it further includes: after extracting rhizoma anemarrhenae with a solvent, concentrating and drying the resultant extracting solution, to obtain the rhizoma anemarrhenae extract existing in a form of extractum or dry powder.

In some embodiments of the present disclosure, the step of enzymatically transforming rhizoma anemarrhenae extract with a compound enzyme of xylanase and cellulase includes:

incubating the rhizoma anemarrhenae extract and the compound enzyme of xylanase and cellulase in a solution to obtain the timosaponin enzymatically transformed product;

Preferably, the ratio of xylanase to cellulase in the compound enzyme is 5:1-1:3, wherein the ratio of xylanase to cellulase is preferably 2:1, and wherein the activity of xylanase is 300 U/mg, and the activity of cellulase is 50 U/mg.

In some preferred embodiments of the present disclosure, the solution used for incubation is a buffer solution;

Preferably, the solution used for incubation includes an acetic acid-sodium acetate buffer solution;

More preferably, pH of the buffer solution ranges from 3.79 to 4.95 (for example, but not limited to, 4.0, 4.2, 4.5, 4.7, etc.).

In some preferred embodiments of the present disclosure, the incubation is carried out at a temperature of 35-55 ° C. (for example, it may be, but not limited to, 40, 45, 50 ° C., etc.) for 1-3 h (for example, it may be, but not limited to, 2 h, etc.).

In some embodiments of the present disclosure, the step of purifying the timosaponin enzymatically transformed product includes: adding timosaponin enzymatically transformed product into a certain amount of alkaline test solution, stirring them for 3 hours to mix them well, standing for a certain length of time, discarding supernatant and retaining the precipitate. A certain amount of ethanol solution is added into the precipitate, and then stirred for 1 hour to dissolve the requisite substances. After standing for a certain length of time, the precipitate is discarded. The supernatant is retained and evaporated of recover ethanol is recovered until there is no ethanol smell. The supernatant is dried under vacuum to obtain the timosaponin enzymatically transformed product.

In this embodiment, the alkaline test solution may be ammonia solution, sodium hydroxide solution, etc., wherein 10% sodium hydroxide solution is optimal, of which the amount used is twice the weight of the timosaponin enzymatically transformed product. The concentration of the ethanol solution is 70%-100%, and anhydrous ethanol or 95% ethanol is preferred.

In this embodiment, what is obtained is the timosaponin enzymatically transformed product with timosaponin AIII as a main effective ingredient, and in the timosaponin enzymatically transformed product, the content of timosaponin AIII is about 55-65%, and the content of timosaponin BII is less than 1%.

In some preferred embodiments of the present disclosure, the timosaponin enzymatically transformed product can be used for inhibiting one or more skin superficial fungi of candida albicans, cryptococcus neoformans, trichophyton rubrum, trichophyton mentagrophytes, and microsporum gypseum, and can be further used as an active ingredient for preparation of drugs for inhibiting skin superficial fungi, and for treatment of superficial mycoses such as onychomycosis, tinea manuum, tinea pedis, tinea capitis, tinea corporis, tinea cruris, and onychomycosis.

In some preferred embodiments of the present disclosure, the timosaponin enzymatically transformed product can be used as an active ingredient, and mixed with a cosolvent to obtain corresponding pharmaceutical dosage forms, such as lotion, liniment, ointment and film coating agent.

Compared with the existing technology, the present disclosure has following beneficial effects:

In the present disclosure, as an active pharmaceutical ingredient for inhibiting skin superficial fungi, the timosaponin enzymatically transformed product shows the minimum inhibitory concentration of 4 mg/L against fungi, which is obviously superior to total timosaponin in the bacteriostatic capability, and has a certain application prospect in the aspect of treating skin superficial mycoses.

In addition, concerning the technical solution in which the compound enzyme of xylanase and cellulase is applied, the present disclosure further has following beneficial effects:

the preparation of timosaponin AIII through direct enzymolysis of the rhizoma anemarrhenae alcohol extract is realized, avoiding the macroporous resin purification process of timosaponin BII from the rhizoma anemarrhenae alcohol extract . Meanwhile, the compound enzyme of xylanase and cellulase instead of β-glucosidase helps to improve the enzymolysis transformation rate of timosaponin AIII is improved to 75.0%-80.0%.The followed purification process of timosaponin AIII is simplified by washing with the alkaline solution and the ethanol solution, thus the production cost is reduced by 70%. The content of timosaponin AIII in the timosaponin enzymolysis product is up to 55-65%, which can meet the requirements of research and drug development for treating skin superficial mycoses.

### Example 1:

### Preparation of Timosaponin Enzymatically Transformed Product

(1) Preparation of ethanol extract of rhizoma anemarrhenae:

60 g of medicinal material rhizoma anemarrhenae (or decoction pieces) was added to 50% ethanol (6 times the amount of the rhizoma anemarrhenae), followed by heating reflux. A boiling state was maintained. The extraction was carried out for 2 times and 2 h each time. After filtration, the extracting liquors were combined and evaporated to recover ethanol. Then the crude extract solution was dried through water bath and vacuum drying under reduced pressure at 60° C. overnight to obtain the rhizoma anemarrhenae ethanol extract.
(2) Preparation of timosaponin enzymatically transformed product

4 g of the ethanol extract of rhizoma anemarrhenae (equivalent to 6 g of medicinal material rhizoma anemarrhenae) and 4 g of δ-glucosidase (50 U/mg) were placed in a conical flask with a plug, to which 200 mL of acetic acid-sodium acetate buffer solution (pH of 4.0) was added. The mixture was placed in a vapor bathing constant temperature vibrator shaking for 2 h. The resultant was dried in a vacuum drying oven at 60° C. under reduced pressure, to obtain the timosaponin enzymatically transformed product.
(3) Measuring content of the timosaponin AIII in the timosaponin enzymolysis product:

Chromatographic condition: a Platisil C18 chromatographic column (4.6 mm ×250 mm, 5 μm); mobile phase: gradient elution with acetonitrile (A)—water (B) (0 min, 25% A; 8 min, 25% A; 9 min, 75% A; 15 min, 75% A; 16 min, 25% A; 20 min, 25% A); flow rate: 1 mL/min; a sample injection amount was 20 μL; column temperature: 30° C., the temperature of an evaporative light scattering detector was 60 ° C., and a flow rate of atomizing gas was 1.5 L/min.

10.00 mg of timosaponin AIII control substance was precisely weighed, placed in a 10 mL volumetric flask, sufficiently dissolved with methanol, to volume, and mixed well to obtain a stock solution of timosaponin AIII control substance with a concentration of 1.00 mg/mL, respectively. Control substance solutions with timosaponin AIII concentrations of 49.35, 98.70, 246.8, 493.5, 789.6, and 987.0 mg/L were prepared, and respectively injected into a high performance liquid chromatograph system. A standard curve was drawn by taking common logarithm of the concentration of the timosaponin AIII control substance as abscissa (X), and taking logarithmic values of respective corresponding peak areas as vertical coordinate (Y), and a regression equation was calculated. 10 mg of the enzymolysis product was precisely weighed, placed in a 25 mL volumetric flask, ultrasonically dissolved with methanol to volume, filtered by a 0.45 μm microporous filter membrane, and injected into a liquid chromatograph system to measure the content of timosaponin AIII.

Result shows that the content of the timosaponin AIII in the timosaponin enzymatically transformed product is 52.47%±0.32% (RSD =0.5%).

### Example 2:

### Optimal Selection of Enzyme in the Timosaponin Enzymolysis Process (1) Preparation of ethanol extract of rhizoma anemarrhenae:

20 kg of rhizoma anemarrhenae was added to 50% ethanol (6 times the amount of the rhizoma anemarrhenae), followed by heating reflux. The extraction was carried out 2 times with 2 hours each time. After filtration, the extracting liquors were combined and evaporated to recover ethanol until the solution volume reach 10L to obtain the rhizoma anemarrhenae extract, which was weighed.

(2) Preparation of timosaponin enzymatically transformed product:

Enzymolysis: 16 g of the above rhizoma anemarrhenae extract was added into 400 mL of an acetic acid-sodium acetate buffer solution (pH 4.5).9.6 g of enz'yme (a compound enzyme of xylanase and cellulase: 5:1, 4:1, 3:1, 2:1, 1:1, 1:2, 1:3, β-glucosidase) was added into the buffer solution and the mixture was stirred at constant of 55° C. to react for 3 hours.

Stirring to remove impurities: 35 mL of 10% sodium hydroxide solution was added into the mixture and then stirred for 3 hours. After standing for a certain length of time, the supernatant was discarded, and precipitate was retain. 640 mL of 95% ethanol was added into the precipitate, and the mixture was stirred for 1 hour to dissolve the requisite substances. After standing for a certain length of time, the precipitate was discarded. Recovering ethanol: the supernatant was retained and evaporated to recover ethanol until there was no ethanol smell. The resultant was dried at 60° C. under vacuum to obtain the timosaponin enzymatically transformed product, and an enzymolysis transformation rate of the timosaponin Alll therein was measured.
(3) Measuring Content of the Timosaponin Alll in the timosaponin enzymolysis product:

Chromatographic condition: a Platisil C18 chromatographic column (4.6 mm ×250 mm, 5 μm); mobile phase: gradient elution with acetonitrile (A)—water (B) (0 min, 25% A; 8 min, 25% A; 9 min, 75% A; 15 min, 75% A; 16 min, 25% A; 20 min, 25% A); flow rate: 1 mL/min; a sample injection amount was 20 μL; column temperature: 30° C., the temperature of an evaporative light scattering detector was 60° C., and a flow rate of atomizing gas was 1.5 L/min.

10.00 mg of timosaponin AIII control substance was precisely weighed, placed in a 10 mL volumetric flask, sufficiently dissolved with methanol, diluted with methanol to volume, and mixed well to obtain a stock solution of timosaponin AIII control substance with concentration of 1.00 mg/mL, respectively. Control substance solutions with timosaponin AIII concentrations of 49.35, 98.70, 246.8, 493.5, 789.6, and 987.0 mg/L were prepared, and respectively injected into a high performance liquid chromatograph system. A standard curve was drawn by taking common logarithm of the concentration of the timosaponin AIII control substance as abscissa (X), and taking logarithmic values of respective corresponding peak areas as vertical coordinate (Y), and a regression equation was calculated. 10 mg of the enzymolysis product was precisely weighed, placed in a 25 mL volumetric flask, ultrasonically dissolved with methanol to volume filtered by a 0.45 μm microporous filter membrane, and injected into a liquid chromatograph system to measure the content of timosaponin AIII. The enzymolysis transformation rate of the timosaponin AIII in the timosaponin enzymolysis process was calculated.

TABLE 1
Result of Optimal Selection of Enzyme in
Timosaponin Enzymolysis Process

| | Enzymolysis Transformation Rate |
|---|---|
| | (%) of Timosaponin AIII (n = 3) |
| compound enzyme (5:1) | 69.3 ± 3.7 |
| compound enzyme (4:1) | 73.2 ± 5.6 |
| compound enzyme (3:1) | 71.2 ± 8.9 |
| compound enzyme (2:1) | 77.3 ± 1.7 |
| compound enzyme (1:1) | 73.1 ± 4.6 |
| compound enzyme (1:2) | 72.5 ± 3.2 |
| compound enzyme (1:3) | 71.1 ± 2.8 |
| β-glucosidase | 69.8 ± 3.2 |

Result shows that the enzymolysis transformation rate of timosaponin AIII in timosaponin by us if the compound enzyme of xylanase and cellulase (2:1) is the highest, being 77.3±1.7, therefore, the following experiments were carried out with the compound enzyme of xylanase and cellulase (2:1).

### Example 3

### Preparation of Timosaponin Enzymatically Transformed Product

(1) Preparation of ethanol extract of rhizoma anemarrhenae:

To 20 kg of rhizoma anemarrhenae was added to 50% ethanol (6 times the amount of the rhizoma anemarrhenae), followed by heating reflux. The extraction was carried out 2 times with 2 hours each time. After filtration, the extracting liquors were combined and evaporated to recover ethanol until the solution volume reach 10 L, to obtain the rhizoma anemarrhenae extract, which was weighed.

(2) Preparation of timosaponin enzymatically transformed product:

Enzymolysis: the above rhizoma anemarrhenae extract was taken, and added into 400 L of an acetic acid-sodium acetate buffer solution (pH 4.5), 9.6 kg of a compound enzyme of xylanase and cellulase was added into the buffer solution and the mixture was stirred at constant 55° C. to react for 3 hours.

Stirring to remove impurities: 35 L of 10% sodium hydroxide solution was added into the mixture and then stirred for 3 hours .After standing for a certain length of time ,the supernatant was discarded and precipitate was retain. 640 L of 95% ethanol was added into the precipitate, and the mixture was stirred for 1 hour to dissolve the requisite substances. After standing for a certain length of time, the precipitate was discarded.

Recovering ethanol: the supernatant was retained, and evaporated to recover ethanol was recovered until there was no ethanol smell. The resultant was dried at 60° C. under vacuum to obtain the timosaponin enzymatically transformed product. The content of the timosaponin AIII in the timosaponin enzymatically transformed product was 65.32%±0.15% (RSD=0.2%).

The following formulations 1-4 containing the timosaponin enzymatically transformed product were prepared using the timosaponin enzymatically transformed product prepared in Example 1 or 3 as raw material:

### Preparation Example 1

### Ointment of Timosaponin Enzymatically Transformed Product

15 g of white petrolatum and 1.5 g of lanolin were mixed and stirred, then 0.2 g of the timosaponin enzymatically transformed product (containing 0.1 g of timosaponin AIII, equivalent to 4.06 g of medicinal material rhizoma anemarrhenae) was added. The mixture was stirred uniformly to obtain an ointment formulation of the timosaponin enzymatically transformed product.

Preparation Example 2: Film Coating Agent of Timosaponin Enzymatically Transformed Product 0.2 g of the timosaponin enzymatically transformed product (equivalent to 4.06 g of medicinal material rhizoma anemarrhenae) were weighed, added into 10 mL of glycerin, and dissolved ultrasonically. 6 g of polyvinyl alcohol was taken, and added into 60 mL of water for swelling at 90 ° C. The two solutions above were mixed well, and added with water to 100 mL, followed by high-pressure sterilization to obtain the film coating agent of the timosaponin enzymatically transformed product, wherein the concentration of the timosaponin enzymatically transformed product was 2 mg/mL (with 1 mg/mL timosaponin AIII).

Preparation Example 3: Lotion of Timosaponin Enzymatically Transformed Product 0.2 g of the timosaponin enzymatically transformed product (equivalent to 4.06 g of medicinal material rhizoma anemarrhenae) was put into a mortar, to which glycerin and an appropriate amount of distilled water were added. They were ground to a paste. Water was gradually added until the concentration of the timosaponin enzymatically transformed product was 2 mg/mL (with 1 mg/mL timosaponin AIII), and they were mixed well to obtain the lotion of the timosaponin enzymatically transformed product.

Preparation Example 4: Liniment of Timosaponin Enzymatically Transformed Product 0.2 g of timosaponin enzymatically transformed product (equivalent to 4.06 g of medicinal material rhizoma anemarrhenae) was added into ethanol and dissolved by stirring. An appropriate amount of laurocapram and glycerol were added, then ethanol was added to make the total volume 100 mL. They were mixed well to obtain the liniment of the timosaponin enzymatically transformed product, wherein the concentration of the timosaponin enzymatically transformed product was 2 mg/mL (with 1 mg/mL timosaponin AIII).

Comparative Preparation Example 1: Film Coating Agent of Timosaponin AIII 1 g of chitosan was weighed, and added into 20 mL of water containing 5% glacial acetic acid to swell. 1 g of polyvinyl alcohol was added into 10 mL of water to swell at 90° C. The chitosan and the polyvinyl alcohol above were mixed well according to a proportion of 1:1, followed by high-pressure sterilization for later use.

10.00 mg of timosaponin AIII was precisely weighed, and placed in a 5 mL volumetric flask, dissolved with methanol to volume, thus preparing a 2 mg/mL methanol solution. 100.00 mg DSPC and DSPE-PEG2000 were precisely weighed, and placed in a 1 mL volumetric flask, respectively. Then were dissolved with chloroform to volume, to prepare 100 mg/mL DSPC chloroform solution and 100 mg/mL DSPE-PEG2000 chloroform solution, respectively. An appropriate amount of the above solutions of DSPC, DSPE-PEG2000, and TAIII were taken according to a proportion of 8:2:1, placed in an egg-shaped bottle and mixed well. The mixture was subjected to rotary evaporation under reduced pressure at 60° C. to form a uniform film on a wall of the egg-shaped bottle. The egg-shaped bottle was stored overnight in a desiccator. The egg-shaped bottle was taken out the next day and 5 mL of distilled water was added followed by hydration at 60° C. for 1 h. The mixture was then sonicated in a water bath until it is transparent, and then the product was obtained and stored in a refrigerator at 4° C.

5 mL timosaponin AIII lipid was taken and mixed uniformly with the mixture of chitosan and polyvinyl alcohol prepared above at equal amount, to obtain the film coating agent of timosaponin AIII, wherein the concentration of the timosaponin AIII was 1 mg/mL.

### Comparative Preparation Example 2

### Ointment of Timosaponin AIII

15 g of white petrolatum and 1.5 g of lanolin were weighed and mixed well by stirring. Then 0.1 g of timosaponin AIII (containing 0.1 g of timosaponin AIII, equivalent to 4.06 g of medicinal material rhizoma anemarrhenae) was added, and the mixture was stirred uniformly to obtain an ointment formulation of the timosaponin AIII.

### Comparative Preparation Example 3

### Ointment of Ethanol Extract of Rhizoma Anemarrhenae

15 g of white petrolatum and 1.5 g of lanolin were weighed and mixed well by stirring. 2.7 g of ethanol extract of rhizoma anemarrhenae (equivalent to 4.06 g of medicinal material rhizoma anemarrhenae) (obtained from the method in Example 1) was added, and they were stirred uniformly to obtain an ointment formulation of the ethanol extract of rhizoma anemarrhenae.

### Experimental Example 1

### Research on in vitro Antifungal Effect of Timosaponin Enzymatically Transformed Product

(1) Preparation of a Bacterial Suspension

Spherical bacteria such as cryptococcus neoformans and candida albicans were taken, and activated with YEPD culture solution, and then the concentration of the bacteria solution was adjusted with RPM11640 culture solution to 1×10³˜5 ×10³ CFU/mL.

Trichophyton rubrum, microsporum gypseum, and trichophyton mentagrophytes were activated with a Sabouraud glucose agar culture medium, and the concentration of spores was adjusted with an RPM11640 culture solution to 1×10³5×10³ CFU/mL.

(2) Test Solutions of Bacteriostatic drug

The ethanol extract of rhizoma anemarrhenae (obtained by the method of Example 1) was dissolved in phosphate buffer solution and the concentration of the ethanol extract of rhizoma anemarrhenae was adjusted to 320 mg/mL, 64 mg/mL, 32 mg/mL, and 6.4 mg/mL to prepare test solutions.

The timosaponin enzymatically transformed product (obtained by the method of Example 1) was dissolved in phosphate buffer solution and the concentration of the timosaponin enzymatically transformed product was adjusted to 27 mg/mL, 13.5 mg/mL, and 1.35 mg/mL to prepare test solutions .The concentration of the timosaponin AIII was adjusted with a phosphate buffer solution to 1.5 mg/mL and 150 mg/L to prepare test solutions.

(3) Inhibitory Effect of the Timosaponin Enzymatically Transformed Product on Candida Albicans

5 pL of a bacteria solution of candida albicans (the concentration of the bacteria solution was 1×10⁴ CFU/mL) was sucked, and uniformly coated on the surface of culture medium with a sterile coating rod. Two sterile Oxford cups were lightly placed on each culture plate at an interval of 20 mm. 150 μL of test drug was injected into each Oxford cup. Because of the low solubility of the drugs had I in water, DMSO and methanol were applied to dissolve the drugs. Therefore, 50% DMSO, 5% DMSO, 50% methanol, and 5% methanol solution in an equal volume were added to Oxford cup as control group.

Each bacteriostatic flat plate was placed in a biochemical incubator, and cultured at 30° C. for 48 hours, and the diameter of a bacteriostatic zone was measured. Result shows that the ethanol extract of rhizoma anemarrhenae had a certain inhibitory effect on candida albicans under a relatively high concentration (320 mg/mL), and the diameter of the bacteriostatic zone was 6 mm; after enzymatic hydrolysis, the timosaponin enzymatically transformed product showed greatly improved inhibitory effect on candida albicans, and the diameters of the bacteriostatic zones were respectively 16 mm and 12 mm under the concentrations of 27 mg/mL and 13.5 mg/mL. Therefore, the inhibitory effect of the timosaponin enzymatically transformed product on candida albicans is obviously stronger than that of the ethanol extract without enzymolysis.

(5) Inhibitory effect of ethanol extract of rhizoma anemarrhenae and timosaponin enzymatically transformed product on five kinds of common fungi

In the present experiment, the minimum inhibitory concentrations of the ethanol extract of rhizoma anemarrhenae and the timosaponin enzymatically transformed product against five common pathogenic fungi were detected by through a trace liquid-based dilution method recommended by the Clinical and Laboratory Standards Institute and the Chinese pharmacopoeia. Fluconazole was used as a positive control drug.

A specific experimental method is as follows: candida albicans, cryptococcus neoformans, trichophyton rubrum, trichophyton mentagrophytes, and microsporum gypseum were inoculated to a 96-well plate, respectively. The drug with different concentrations was added to the culture, wherein final concentrations of the drug were 64 mg/L, 32 mg/L, 16 mg/L, 8 mg/L, 4 mg/L, 2 mg/L, 1 mg/L, and 0.5 mg/L, respectively. Fungi treated without drug was used as positive control. The candida albicans was cultured at 35° C. for 24 hours while the cryptococcus neoformans was cultured at 35° C. for 72 hours and the trichophyton rubrum, the trichophyton mentagrophytes, and the microsporum gypseum were cultured at 30° C. for 7 days. The OD value of each well under the wavelength of 630 nm was measured using an microplate reader. The lowest drug concentration with an OD value reduced by 80% or more compared with the positive control wells was considered as MIC8o, namely the drug concentration inducing 80% inhibition against fungus growth was inhibited by 80%. When the MIC₈₀ value of the drug was higher than the maximum concentration of 64 mg/L, “>64 mg/L” was recorded.

Result of bacteriostatic experiments shows that timosaponin AIII had relatively good activity on the filamentous dermatophytes trichophyton rubrum, microsporum gypseum, and trichophyton mentagrophytes, and MIC is4 mg/L. The ethanol extract of rhizoma anemarrhenae and the timosaponin enzymatically transformed product both show different inhibitory activities to the 5 fungus in vitro. Enzymatic hydrolysis significantly enhanced the inhibitory activity of the ethanol extract of rhizoma anemarrhenae, and the MIC of timosaponin enzymatically transformed product is 4 mg/L (shown in Table 1).

TABLE 1
In vitro Bacteriostatic Activity (MIC, mg/L) of Ethanol Extract and
Enzymatically Transformed Product of Rhizoma Anemarrhenae on 5 Fungus

| | | | Cryptococcus | Microsporum | Trichophyton | trichophyton |
|---|---|---|---|---|---|---|
| | C. albicans | C. albicans | neoformans | gypseum | rubrum | mentagrophytes |
| Test Drug | SC5314 | Y0109 | 32609 | mzc | tia | t5a |
| ethanol extract | 32 | 16 | 16 | >64 | >64 | 32 |
| of rhizoma | | | | | | |
| anemarrhenae | | | | | | |
| timosaponin | 4 | 4 | 4 | 4 | 4 | 4 |
| enzymatically | | | | | | |
| transformed | | | | | | |
| product | | | | | | |
| timosaponin | 4 | 4 | 4 | 2 | 2 | 2 |
| Alll | | | | | | |
| posaconazole | 0.5 | 1 | 0.25 | 0.25 | 2 | 0.25 |
| fluconazole | 0.5 | 1 | 1 | 8 | 2 | 32 |

Experimental Example 2: Research on in vivo Antifungal Effect of Timosaponin Enzymatically Transformed Product

(1) Preparation of a Bacterial Suspension

The experimental standard strains were inoculated for at least 2 times of activation (candida albicans and cryptococcus neoformans were cultured at 35° C. for one week; trichophyton mentagrophytes, trichophyton rubrum, and microsporum gypseum were cultured at 26° C. for one week) before the experiment to ensure purification and viability of colony. Before the experiment, the concentration of the bacteria solution of candida albicans and cryptococcus neoformans was adjusted with a phosphate buffer solution to 1×10^{4-1×}10⁵ CFU/mL; For other superficial fungi, an appropriate amount of phosphate buffer solution was added into the Sabouraud glucose agar slant The bacterial colony was slightly rubbed by a sterile glass rod to make spores and broken hyphae dissociative in the buffer solution. The concentration was adjusted to 1×10⁴˜1×10⁵ CFU/mL including spores and hyphae (1 CFU was calculated based on separated hyphae or one spore) by counting through a hemocyometer added to. The concentration of trichophyton mentagrophytes was adjusted with a phosphate buffer solution to (1˜5)×10⁶ CFU/mL.
(2) Establishment of Animal Models

The long hairs on two sides of the spine of back on healthy mice were sheared off using surgical scissors, and then trimmed using an electric shaver to provide smooth hairless areas of 4cm×5 cm .After 24 hours, the hairless areas were disinfected using 75% alcohol. When the hairless areas were dry, the skin was slightly polished using fine sandpaper, to prepare a rough surface with slight bleeding. Then the prepared fungus suspension was uniformly coated on the hairless skin using a cotton swab, and then rubbed emphatically. The wound skin was exposed in air, and observed once a day. After 3-5 days, the local infection was obviously formed, and verified as positive by the mycology examination (direct microscopic examination and culture) indicating the successful infection.
(3) Animal Grouping and Administration

After local skin of a mouse was sterilized with alcohol, a scraper was cauterized to be sterilized and then cooled, a little scale was scraped from the back, and placed on a Sabouraud culture tube and a clean glass slide respectively, a drop of 10% KOH was added on the glass slide, and a cover glass was covered, followed by slight heating on flame until bubbles just appeared at the position of the sample on the glass slide, then the cover glass was slightly pressed, hypha could be observed under an optical microscope, and the culture tube was placed in a 26° C. incubator to be cultured for one week.

After the mice were inoculated with the fungus for 14 days, the successfully infected mice were randomly divided into 5 groups, i.e., rhizoma anemarrhenae ethanol extract ointment group, timosaponin enzymatically transformed product ointment group, timosaponin AIII ointment group, ointment base group, and positive control group (Miconazole Nitrate Cream, also called as Daktarin Cream. Each group involves 10 mice and 20 infected areas (all the ointments used prepared as mentioned above).

Each infected area was coated with drugs at a dosage of 0.2 g 2 times per day and lasting for 2 consecutive weeks, and mycology microscopic examination and skin lesion observation were carried out every 2 days. The evaluation criteria of the curative effect were 2 grades: cured, if the skin lesion faded away, and the fungi microscopic examination was negative 2 times consecutively; and invalid, if the skin lesion did not fade away, and the fungi microscopic examination was positive.

When the timosaponin enzymatically transformed product ointment group, the timosaponin AIII ointment group and the Daktarin group were administrated for 5 days, papules and erythema on the edge of scale gradually faded away, and the color gradually faded. When the drug was administrated for 1 week, scales of some the mice fell off with edema disappeared, and the skin where the scales were fell off was well repaired and recovered to be flat and smooth .The skin of the infected area of the mice was close to normal skin at 2 weeks after administration, and newborn hairs appeared. After the ethanol extract of rhizoma anemarrhenae ointment group was administrated for 2 weeks, only part of the mouse showed well repaired and smooth skin. The wound skin of the infected area in the ointment base control group was not improved during the observation period.

The treatment effect of each group is as shown in the following Table 2:

TABLE 2
Treatment Effect of Timosaponin Enzymatically
Transformed Product Ointment on Mice
Infected with Trichophyton Mentagrophytes

| | Number of In- | 1-week Cure | 2-week Cure |
|---|---|---|---|
| Group | fected Areas | Rate (%) | Rate (%) |
| timosaponin extract | 20 | 10 | 30 |
| ointment group | | | |
| timosaponin AIII | 20 | 50**, ^{##} | 85**, ^{##} |
| ointment group | | | |
| timosaponin | 20 | 50**, ^{##} | 90**, ^{##} |
| enzymatically | | | |
| transformed product | | | |
| ointment group | | | |
| ointment base group | 20 | 0 | 10 |
| Daktarin group | 20 | 40**, ^{##} | 85**, ^{##} |

**means P < 0.01 compared with the ointment base group;
^{##}means P < 0.01 compared with the rhizoma anemarrhenae extract ointment group

As show in Table 2 above, the 2-week cure rates of the timosaponin enzymatically transformed product ointment group and the timosaponin AIII ointment group for the local skin infection of trichophyton mentagrophytes in mice are 90% and 85%. The 2-week cure rate of the Daktarin group was 85%, and there is no significant difference between the two groups (P>0.05).

The 2-week cure rate of the rhizoma anemarrhenae ethanol extract ointment group was 30%, which is obviously lower than those of the timosaponin enzymatically transformed product ointment group and the timosaponin AIII ointment group (P<0.01). It indicates that the treatment effects of the timosaponin enzymatically transformed product ointment and the timosaponin AIII ointment are equivalent to Daktarin on skin tinea disease. The timosaponin enzymatically transformed product and the timosaponin AIII show superior advantages than the ethanol extract of rhizoma anemarrhenae in the development and application of drugs for resisting skin superficial mycoses.

Although the present disclosure has been illustrated and described with specific embodiments, it should be aware that many other alterations and modifications can be made without departing from the spirit and scope of the present disclosure. Therefore, it means that the attached claims cover all these changes and modifications within the scope of the present disclosure.

### INDUSTRIAL APPLICABILITY

In the present disclosure, as an active pharmaceutical ingredient for inhibiting skin superficial fungi, the timosaponin enzymatically transformed product has the minimum inhibitory concentration of 4 mg/L against fungi, which is obviously superior to total timosaponin in the bacteriostatic capability, and has a certain application prospect in the aspect of treating skin superficial mycoses.

What is claimed is:

1. Use of a timosaponin enzymatically transformed product in preparation of drugs for inhibiting skin superficial fungi, wherein the timosaponin enzymatically transformed product is prepared by enzymatically transforming rhizoma anemarrhenae extract with β-glucosidase to obtain the timosaponin enzymatically transformed product.

2. The use according to claim 1, wherein the rhizoma anemarrhenae extract is prepared mainly by extracting a rhizoma anemarrhenae with a solvent to obtain the rhizoma anemarrhenae extract, wherein the rhizoma anemarrhenae comprises one or two of medicinal material rhizoma anemarrhenae and rhizoma anemarrhenae decoction pieces; and
the solvent comprises one of ethanol, aqueous ethanol and water.

3. The use according to claim 1, wherein the enzymatically transforming rhizoma anemarrhenae extract with β-glucosidase comprises:
incubating the rhizoma anemarrhenae extract and β-glucosidase in a solution to obtain the timosaponin enzymatically transformed product.

4. The use according to claim 3, wherein the solution is a buffer solution, wherein the buffer solution comprises an acetic acid-sodium acetate buffer solution.

5. The use according to claim 4, wherein pH of the buffer solution is 3.79-4.95.

6. The use according to claim 3, wherein the incubating is carried out at a temperature of 35-55° C. for 1-3 h.

7. The use according to claim 1, wherein a main ingredient of the rhizoma anemarrhenae extract is timosaponin BII, with a content of 5-10%; and
in a resultant timosaponin enzymatically transformed product, a main ingredient is timosaponin AIII, with a content of 40% or more.

8. The use according to claim 1, wherein the skin superficial fungi comprises one or more of candida albicans, cryptococcus neoformans, trichophyton rubrum, trichophyton mentagrophytes, and microsporum gypseum.

9. The use according to claim 1, wherein the drugs for inhibiting skin superficial fungi are in a dosage form comprising one or more of lotion, liniment, ointment and film coating agent.

10. Use of a timosaponin enzymatically transformed product containing high-purity timosaponin AIII in preparation of drugs for inhibiting skin superficial fungi, wherein the timosaponin enzymatically transformed product is prepared by: enzymatically transforming rhizoma anemarrhenae extract with a compound enzyme of xylanase and cellulose, to obtain a timosaponin enzymatically transformed product after stirring to remove impurities with an alkaline solution and stirring to remove impurities with high concentration ethanol.

11. The use according to claim 10, wherein the rhizoma anemarrhenae extract is prepared mainly by: extracting the rhizoma anemarrhenae with a solvent to obtain the rhizoma anemarrhenae extract, wherein the rhizoma anemarrhenae comprises one or two of medicinal material rhizoma anemarrhenae and rhizoma anemarrhenae decoction pieces; and
the solvent comprises one of ethanol, aqueous ethanol and water.

12. The use according to claim 10, wherein the enzymatically transforming rhizoma anemarrhenae extract with a compound enzyme of xylanase and cellulose comprises:
incubating the rhizoma anemarrhenae extract and the compound enzyme of xylanase and cellulose in an acetic acid-sodium acetate buffer solution, wherein a ratio of xylanase to cellulase in the compound enzyme is 5:1-1:3, and the incubating is carried out at a temperature of 35-55° C. for 1-3 h.

13. The use according to claim 10, wherein the timosaponin enzymatically transformed product is purified, which comprises: adding the timosaponin enzymatically transformed product into a certain amount of an alkaline test solution, stirring them for 3 hours to mix them well, standing and discarding supernatant to obtain precipitate; and adding a certain amount of ethanol solution into the precipitate, stirring them for 1 hour to dissolve, standing and discarding precipitate to obtain supernatant, then recovering ethanol until there is no ethanol smell, and drying the supernatant under reduced pressure to obtain the timosaponin enzymatically transformed product.

14. The use according to claim 13, wherein the alkaline test solution is ammonia solution or sodium hydroxide solution, which is in an amount twice of that of the timosaponin enzymatically transformed product in weight, and a concentration of ethanol in the ethanol solution is 70%-100%.

15. The use according to claim 10, wherein in a resultant timosaponin enzymatically transformed product, the content of ingredient timosaponin AIII is 55% or more, and the content of timosaponin BII is less than 1%.

16. The use according to claim 10, wherein the skin superficial fungi comprises one or more of candida albicans, cryptococcus neoformans, trichophyton rubrum, trichophyton mentagrophytes, and microsporum gypseum.

17. The use according to claim 10, wherein the drugs for inhibiting skin superficial fungi are in a dosage form comprising one or more of lotion, liniment, ointment and film coating agent.

18. A drug for inhibiting skin superficial fungi, wherein a raw material of the drug for inhibiting skin superficial fungi comprises timosaponin enzymatically transformed product or timosaponin AIII.
